(19) United States
(12) Patent Application Publication
(10) Pub. No.: US 2025/0144362 A1
(43) Pub. Date: May 8, 2025

(54) SCREEN FOR IMAGE PROJECTION DURING SURGERY AND A PATIENT ASSISTANCE PROJECTION SYSTEM.

(71) Applicant: José Luis MOSSO-VÁZQUEZ, Mexico City (MX)

(72) Inventor: José Luis MOSSO-VÁZQUEZ, Mexico City (MX)

(21) Appl. No.: 18/833,719

(22) PCT Filed: Jan. 26, 2022
(86) PCT No.: PCT/IB2022/050674

§ 371 Date: Jul. 26, 2024

(51) Int. Cl. A61M 21/02; A61M 21/00

(52) U.S. Cl. CPC A61M 21/02; A61M 2021/005

(57) ABSTRACT

A back projection screen that allows the awake patient to move the head and neck freely and yet be able to look at the images that are displayed during surgery, but at the same time allows a surgery crew, in particular an anesthesiologist, to watch the airway passage and the consciousness state of the patient, and to monitor signs including color of the lips, eyes movement, verbal responses, skin sensitivity, and the like, as well as to maneuver for intubation or other procedures without removing the screen during surgery. The back projection screen allows also eye contact and/or face to face interaction, physically or virtually, with the surgeon or other members of the surgery team for effective communication and is suitable for easy cleaning or disinfection from bacteria or viruses as required by operating rooms.

### FIELD OF THE INVENTION

The present invention is related to the field of surgery and medical intervention aid equipment, and more specifically, it is related to a screen for image projection during surgery or medical procedures and to a system for the projection of images during surgery or medical procedures under regional or local anesthesia with the patient awake.

### BACKGROUND OF THE INVENTION

Surgery has generally evolved over the years to incorporate the use of new technologies, and the development of virtual reality, real time communications as a surgeon preforms the surgical operations, and generally the transmission of images in real-time as the surgery happens have deployed over the last two decades.

One specific application of these technologies is the possibility of benefiting not only the surgeon, but the persons that are subject to a surgical procedure in terms of their psychological and physiological reactions, as it has been studied that the state of mind of the patients during surgery often has an impact in the final outcome.

Such effects have been studied by Folen, et. al. (2009, Immersive panoramic video display compared to flat screen display: Psychological and physiological reactions to anger stimuli. at the 14th Annual International CyberTherapy and CyberPsychology, Verbania-Intra, Italy 2009), Mosso, et. al. (2012, Cybertherapy in Medicine-Experience at the Universidad Panamericana, IMSS and ISSSTE Mexico. Virtual Reality in Psychological, Medical and Pedagogical Applications, 229 BoD-Books on Demand), and more recently Sadeghi, et. al. (2020, Current and future applications of virtual, augmented, and mixed reality in cardiothoracic surgery. The Annals of Thoracic Surgery, Elsevier).

However, while the benefits of such aids have been studied and reported, the ability to introduce certain equipment into surgery rooms under sterile conditions, and furthermore, the availability of equipment that may be reused under such sterile conditions, but maintaining practical utility for both, patients and surgeons, has not been solved yet. In spite of the large availability of equipment that is designed as an aid to the surgeon and even for educational purposes, telemedicine or surgery robot controlling, very few developments have been centered in the patient's comfort and psychological wellbeing during the surgery.

For instance, utility model CN213489347U refers to a U Head and face supporting face screen for patient with trigeminal neuralgia through CT guided downlink radio frequency operation, which includes a device specifically designed to be positioned on the face of the patient. The utility model discloses a system to be placed on a patient with trigeminal neuralgia that leads down radio frequency through a head-face portion supporting a face screen with CT, including an arc screen, a hood, a band, a sponge backing plate and shop's piece of cloth. The utility model supposedly allows good circulation of air but the aim is to protect the patient's head and face rather than projecting anything therein. Accordingly, although the device has enough space for fixing a nasal catheter and avoid influencing the surgery, the device is not suitable for projecting images to be seen by the patient and/or the surgeon, and it does not allow an anesthetist to physically monitor the patient through direct sight in order to make an urgent intervention.

There are other systems that are arranged for visualization of images, such as patent application US20210121238 A1 that discloses a visualization system, and a method for ENT (ear-nose-throat) procedures, which shows how it is possible to capture images and connect visualization devices such as glasses to processing means such as a smart phone, which as mentioned before, are destined to the use of the surgeon and to controlling surgical instruments, or even for transmitting the images to other locations, but not to the use by the patient.

There are other technologies for projection of images during surgery, such as the devices described in documents WO2019194174, EP3018900 A4 or JP2005292453 A, which represent solutions for the projection of images inside a surgery room in terms of the operation for the continued projection of images given the needs of movement of the surgeon, light conditions or image processing based on captures and further projection to determined locations on the body of the patient, but they are not oriented to the surface or object where such images are projected, nor are they designed or adapted to allow the patient subject to surgery to watch the images projected by such devices.

Other developments focus on systems, methods and devices which are oriented more to augmented reality and three-dimensional projection of images, the means for processing the same to achieve such projections or goals but that are also oriented to the surgeon's performance, such as patent application US20210121237 A1, U.S. Pat. No. 11,122,250 B2 or US patent application US20170127909 A1.

Regarding the displays as such, some developments provide for systems for observing three-dimensional images without the need for the user to wear specially adapted lenses or glasses, such as the one described in U.S. Pat. No. 7,184,212 B2.

One of the few examples in the prior art with specific focus on allowing the patient to visualize images during the surgery is EP3692416 A1. The Systems and methods described therein allow delivering images to a patient before and/or during a medical procedure in which a patient is translated on a table relative to a gantry, with emphasis in keeping the projected field size during table motion and providing immersive viewing to the patient. The document describes embodiments where the projected field size is maintained by a display system that is secured to the table such that both a projector and a projection screen are fixed relative to the table, and relative to the patient, during translation of the table. Regarding the shown images, the system aims to reduce patient anxiety by using virtual images that are perceived by the patient as residing at a depth that lies beyond the confined spatial region in which the patient resides. The systems therein described have still several practical problems. On one hand, the patient cannot move the head and neck freely and his attention is fixed on the screen, and the patient cannot turn the head easily. Furthermore, the dimensions and position of the screen limits the work of the anesthetist, which main job is to watch the air passage and the consciousness state of the patient, amongst other tasks such as watching the color of the lips, eyes movement and the like, or even to maneuvering for intubation or other procedures due to the lack of space between the face of the patient and the projection screen. Moreover, many times the patients need to have eye contact with the surgeon or other members of the surgery team, and the device does not allow for effective communication with the patient during surgery.

In addition, as previously noted, surgery rooms require strict hygiene measures and devices such as the one described in EP3692416 A1 have many joints and movable elements that are difficult to clean or disinfect from bacteria or viruses. Although it is thought that the sense of isolation helps the patient, the feeling of confinement is increased by tools that are either too close to the face of the patient or directly fixed to the eyes of the patient through googles or glasses that do not allow the patient to be aware of the environment around.

Consequently, there has been sought a solution to solve the above problems of the devices found in the prior art by allowing at the same time the work of the surgical team for monitoring the patient during the surgery and also the possibility of communication with outside physicians or family, and other image visualization in order to reduce anxiety and stress due to the surgery without a sense of confinement.

### Objectives of the Invention

Considering the drawbacks and problems of the displaying techniques during surgery or medical procedures of the prior art, it is an objective of the present invention to provide a screen that allows the patient to move the head and neck freely and yet be able to look at the images and/or videos that are displayed during surgery or medical procedures.

It is another objective of the present invention to provide a screen that allows a surgery or medical crew, in particular an anesthetist, to watch the air passage and the consciousness state of the patient, and to monitor signs including color of the lips, eyes movement, verbal responses, skin sensitivity, and the like.

It is yet another objective of the present invention to provide a screen that allows a surgery or medical crew, in particular an anesthetist, to maneuver for intubation, gastric tube, heart electrodes or other procedures without removing the screen during surgery.

It is a further objective of the present invention to allow eye contact and/or face to face interaction, physically or virtually, with the surgeon, other members of the surgery team or family for effective communication improving patient-surgeon relationship.

It is an additional objective of the present invention to provide a screen that is suitable easy cleaning or disinfection from bacteria or viruses as required by surgery rooms.

It is still another objective of the present invention to provide a system for image display that includes a screen complying with the other objectives, that reduces, fear, isolation anxiety and the level of confinement of a person subject to surgery or medical procedures when the system is in use and allows the person to be aware of the environment surrounding such person and a method for reducing anxiety using the screen.

These and other objectives are achieved through a screen for image projection during surgery or medical procedures and an image projection system incorporating the same in accordance with the principles of the present invention.

### BRIEF SUMMARY OF THE INVENTION

In order to achieve its objectives, under the principles of the present invention it is provided a projection screen comprising:
a laminar body with an arched surface for projection of images that is configured to face the concave side of the arched surface towards a patient, who will see the images therein projected, wherein the arc has a length and angle enough to define a space from the nose of the patient to the center of the arc that is enough to allow a health professional to monitor the patient's signs and symptoms and to maneuver around the patient's mouth and nose to control anesthesia or in case of surgical emergency; and
supporting means support the laminar body and place the projection screen on a surface where the patient lays;

wherein the projection screen is made in one piece, without sharp edges, of a material that is sterilizable, disinfectant and/or susceptible of disinfection, and has an opacity enough to allow the patient and the members of the surgery or medical team to see through the projection screen but also to allow the projection of images on the arched surface to be seen by the patient during surgery or medical procedures.

Another aspect of the present invention is to provide a patient assistance projection system comprising the projection screen according to the principles of the present invention; at least one set of processing means capable of storing, capturing, showing, communicating and/or transmitting images or video; and, at least one projector capable of receiving image or video data from the processing means and conform an image to be shown on the projection screen.

### BRIEF DESCRIPTION OF THE FIGURES

The novel aspects that are characteristic of the present invention shall be set forth clearly in the appended claims. However, some embodiments, features and advantages thereof shall be better understood when read in connection with the appended figures, wherein:

FIG. 1 is a perspective view of an embodiment of the projection screen of the present invention that is configured to be placed on a surface where a patient subject to surgery or medical procedures lays.

FIG. 2 is a front elevation view of the projection screen of FIG. 1.

FIG. 3 is a back elevation view of the projection screen of FIG. 1.

FIG. 4 is a left elevation view of the projection screen of FIG. 1.

FIG. 5 is a right elevation view of the projection screen of FIG. 1.

FIG. 6 is a left elevation view of a second embodiment of the projection screen of the present invention.

FIG. 7 is a right elevation view of the projection screen of FIG. 6.

FIG. 8 is a top view of the projection screen of FIG. 1.

FIG. 9 is a bottom view of the projection screen of FIG. 1.

FIG. 10 is a schematic view of an embodiment of the patient aid projection system of the present invention configured to be used with the projection screen of FIG. 1.

### DETAILED DESCRIPTION OF THE INVENTION

Under the principles of the present invention it is provided a projection screen comprising:
a laminar body with an arched surface for projection of images that is configured to face the concave side of the arched surface towards a patient, who will see the images therein projected, wherein the arc has a length and angle enough to define a space from the nose of the patient to the center of the arc that is enough to allow a health professional to monitor the patient's signs and symptoms and to maneuver around the patient's mouth, nose and airway in case of emergency; and
supporting means support the laminar body and place the projection screen on a surface where the patient lays;

wherein the projection screen is made in one piece, without sharp edges, of a material that is sterilizable, disinfectant and/or susceptible of disinfection, and has an opacity enough to allow the patient and the members of the surgery or medical team to see through the projection screen but also to allow the projection of images on the arched surface to be seen by the patient during surgery or medical procedures.

In a preferred embodiment of the invention, the laminar body is made of a composite of at least two layers, a support layer comprising a transparent polymeric material for providing mechanical support to the projection screen, and a projection layer comprising at least one film with a surface treated to provide the opacity and texture necessary for the back projection of the images. In a particularly preferred embodiment of the invention, the laminar body comprises additionally a protective layer of transparent polymeric material, preferably made of the same material than the support layer, thereby forming a composite where the projection layer is embedded within the support and the protective layers to enhance durability of the projection screen. Preferably, the support layer is located on the concave side of the arched surface of the projection screen.

The arc defining the arched surface of the projection screen of the present invention has preferably an angle between 130 and 250 degrees, with a preferred cord equal to the standard width of a surgery table. In another embodiment of the invention the preferred cord of the arc is as a minimum 30% larger than the maximum expected biacromial diameter-shoulder length—of a person. More preferably, the arc has an angle between 180 and 250 degrees. Furthermore, other embodiments include the possibility of having a different angle of the arc at the front and the back of the projection screen.

Regarding the supporting means, in a preferred embodiment of the projection screen, they are located along the edge of the laminar body at the extremes of the arch defined by its arched surface and extend along the cord of the arch, thereby joining the two arched surface extremes to conform a single piece with the laminar body and to provide a supporting surface for contact with another surface intended to let a patient lay such as a surgery table or a gurney. In a preferred embodiment of the invention, the supporting means comprise a first section that extends from the end point of the laminar body towards the supporting surface and a second section consisting of the supporting surface itself.

The edges conformed by any change of planes in the arched screen are preferably rounded to avoid the conformation of sharp angles that could make it difficult to clean or disinfect.

In another embodiment of the invention, the supporting means define a larger length on one side of the laminar body than the length of the supporting means at the other side of the laminar body, to provide a larger space to allow a health professional to maneuver around the mouth and nose of a patient subject to surgery or medical procedures but also to provide a better angle for the patient and the health professionals to view the projected images. In the particular embodiment where the supporting means comprise a first and second sections, the larger space is defined by the first section.

In a preferred embodiment of the supporting means, it includes holding means, preferably consisting of at least one handle that is formed by an opening on the side of the supporting means to facilitate manipulation of the projection screen.

In another possible embodiment of the invention, the supporting means include shapes to fit the arms of a patient in order to increase the comfort during surgery or medical procedures.

Another aspect of the present invention is to provide a patient assistance projection system comprising the projection screen according to the principles of the present invention; at least one set of processing means capable of storing, capturing, showing, communicating and/or transmitting images or video; and, at least one projector capable of receiving image or video data from the processing means and conform an image to be shown on the back projection screen.

In one embodiment of the present invention, the processing means are selected from cell phones or tablets with image and video capture and transmission capabilities, which are connected to live video contents or have memory stored image or video contents.

The processing means may be connected to the projector by any suitable connection means, preferably selected from cable or wireless communication systems. In a preferred embodiment the projection system includes holders for the processing means and/or the projector, to facilitate manipulation and location of both during surgery or medical procedures, preferably selected from cell-phone holders, tripods, surgery room fixtures or medical device holders.

In a preferred embodiment of the projection system of the invention, the projector is a micro-projector, preferably lightweight, capable of projecting from at least 1 meter to fit the projected content on the arched surface.

The projection system that includes the projection screen under the principles of the present invention reduces anxiety and the level of confinement of a person subject to surgery or medical procedures when the system is in use and allows the person to be aware of the environment surrounding such person.

Under the principles of the present invention it is possible to provide a method for reducing the anxiety level of a patient subject to surgery or a medical procedure comprising the steps of placing a projection screen according under the principles of the present invention; projecting on the projection screen an image or video suitable to reduce the anxiety of the patient; and, subjecting the patient to the surgery or medical procedure.

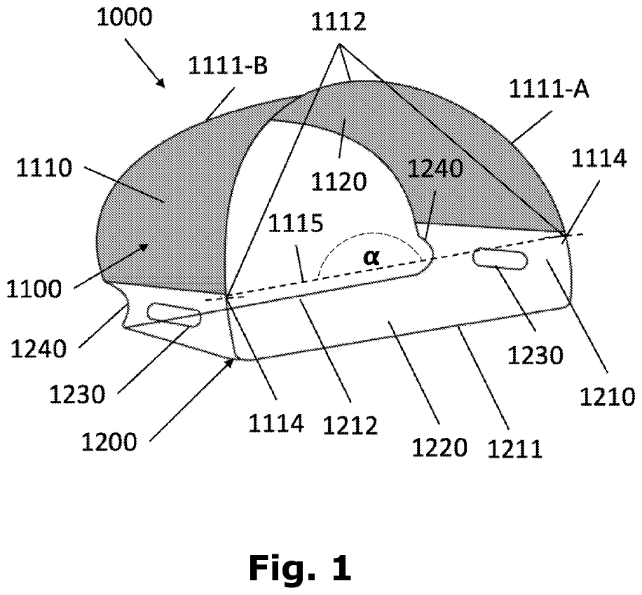

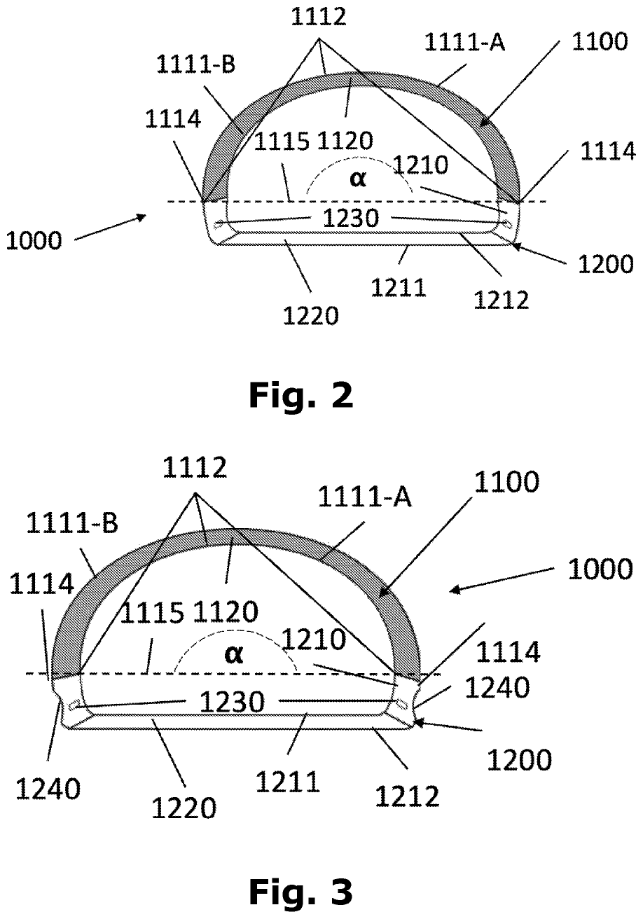

The present invention shall be better understood with reference to thes particular embodiments shown in the appended figures, which are intended to illustrate the principles of the invention but should not be considered as limiting the broader described concepts. As it can be observed in FIGS. 1 to 9, the projection screen (1000) comprises the laminar body (1100) with an arched surface (1110) configured to face the concave side (1120) of the arched surface (1110) towards a patient. The arc (1111) defined by the arched surface (1110) has a length (1112) and angle («) enough to define a space from the nose of the patient to the center of the arc (1111) that is enough to allow a health professional to monitor the patient's signs and symptoms and to maneuver around the patient's mouth and nose in case of emergency. The projection screen includes also supporting means (1200) to support the laminar body (1100) on a surface where the patient lays.

In the embodiments of FIGS. 1 to 9, the supporting means (1200) are located along the edge of the laminar body (1100) at the extremes (1114) of the arch (1111) defined by its arched surface (1110) and extend along the chord (1115) of the arch (1111), thereby joining the two arched surface extremes (1114) to conform a single piece with the laminar body (1100) and to provide a supporting surface (1220) for contact with another surface intended to let a patient lay such as a surgery table or a gurney. The supporting means (1200) comprise a first section (1210) that extends from the end point of the laminar body towards the supporting surface and a second section (1220) consisting of the supporting surface itself.

The edges conformed by the change of planes such as the intersection of the first section (1210) and second section (1220) in the arched screen are rounded to avoid the conformation of sharp angles that could make it difficult to clean or disinfect.

As shown in FIGS. 1 to 9, the supporting means (1200) at the first section (1210) define a larger length (1211) on one side of the laminar body (1100) than the length of the supporting means (1200) at the other side (1212) of the laminar body (1100), to provide a larger space to allow a health professional to maneuver around the mouth and nose of a patient subject to surgery or medical procedures but also to provide a better angle for the patient and the health professionals to view the projected images. The embodiment as shown in the figures also includes holding means (1230) consisting of openings on each side of the supporting means (1200) to facilitate manipulation of the projection screen (1000), and special shapes (1240) to fit the arms of a patient to increase the comfort during surgery or medical procedures.

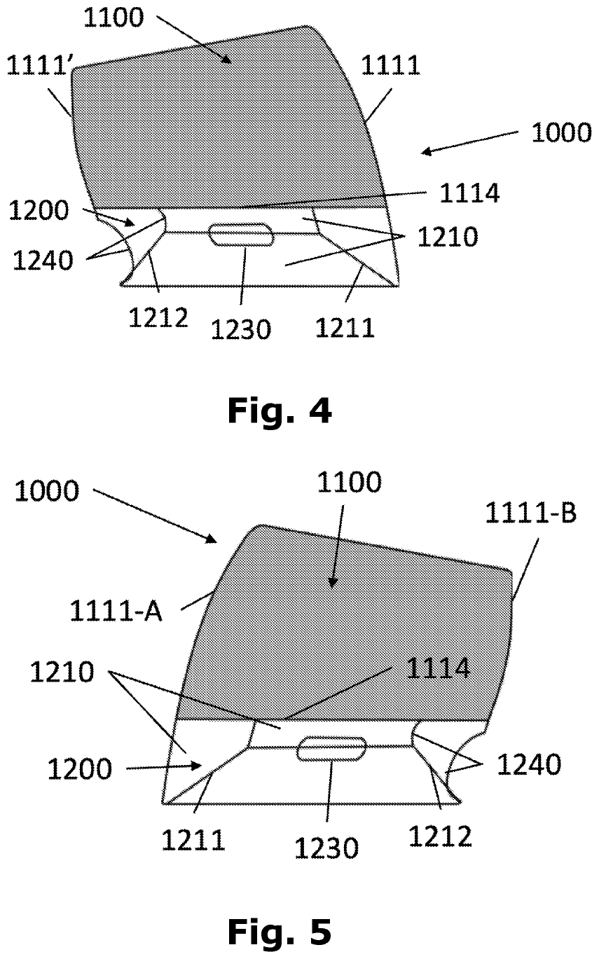

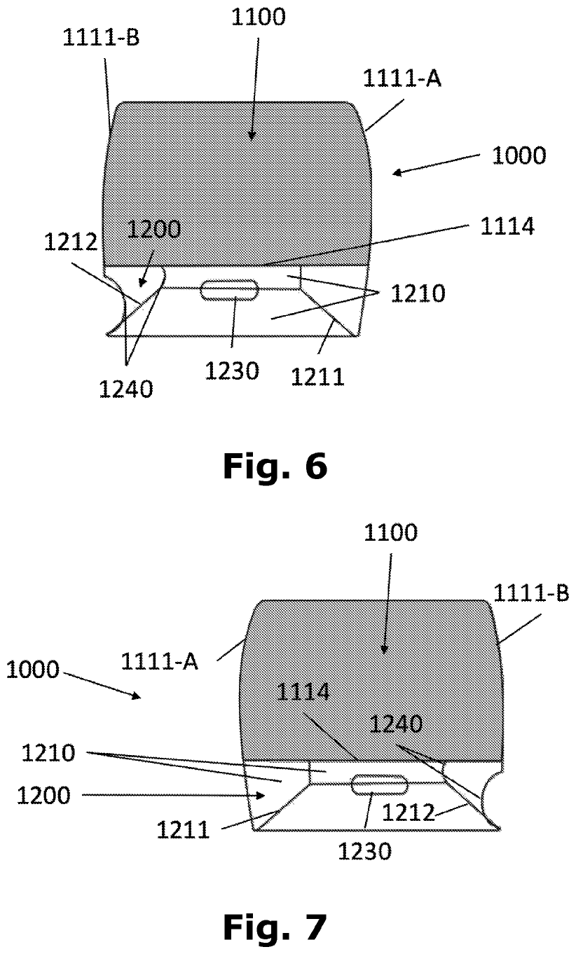

It is noted that FIGS. 4 and 5 show a particular embodiment where the arc (1111) is not perpendicular to the supporting means (1200) in order to provide an inclination for a better view by the patient. However, as observed in FIGS. 5 and 6, the height of the projection screen (1000) may be the same at the front than at the back, or as in the case of FIGS. 4 and 5, differentiated by defining a different angle of the arc (1111-A) at the front than the arc defining the arched surface at the back (1111-B). Accordingly, those skilled in the art will know that based on the principles of the invention other embodiments and combinations of sizes and angles are possible to achieve the objectives of the invention.

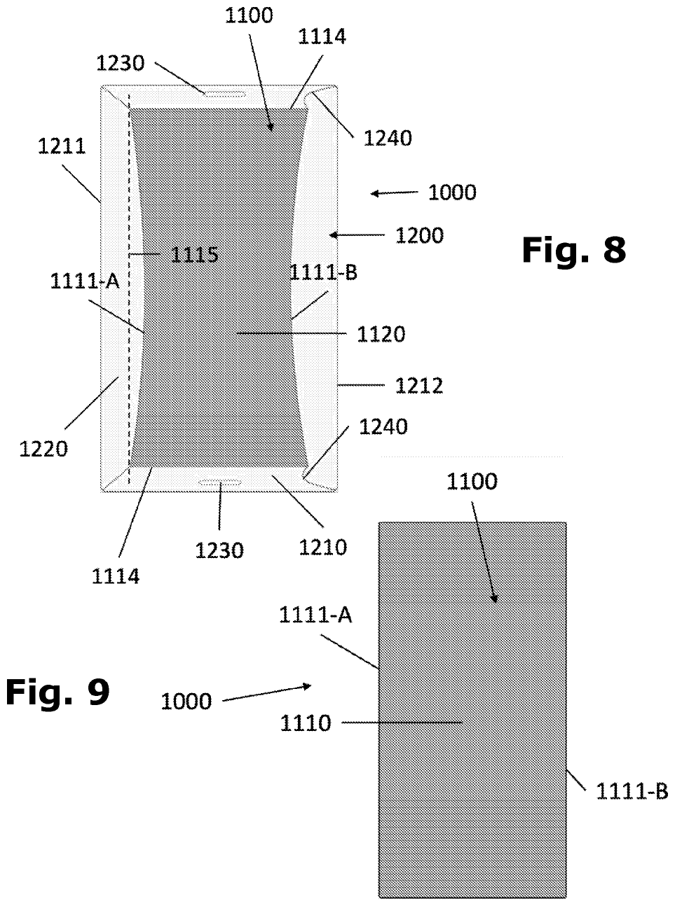

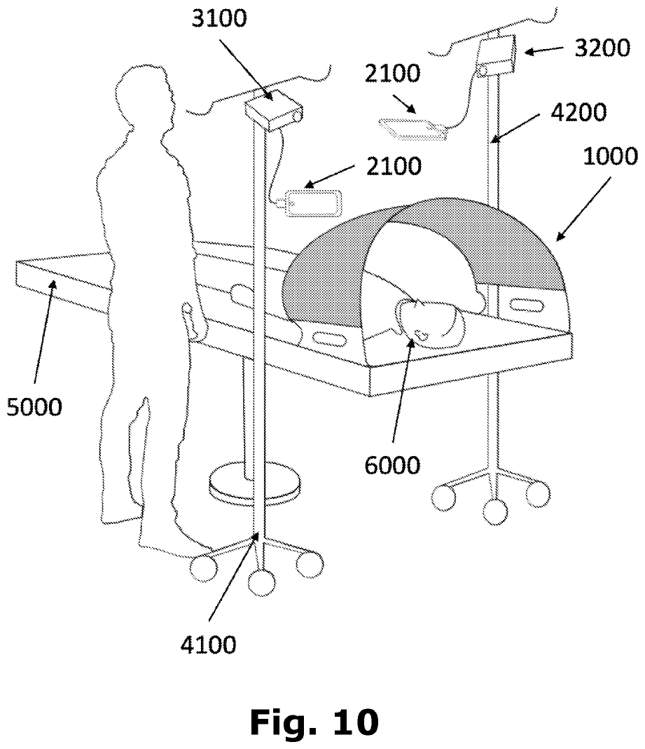

Regarding the system, it will be apparent for those skilled in the art that the configuration of the different elements of the system is not restricted to a specific location of such elements, provided that they are so located to allow projection on the projection screen (1000). The particular embodiment of FIG. 10 shows the projection screen (1000) of FIGS. 1 to 9 in use with the system of the present invention, which comprises a first cell-phone (2100) and a second cell-phone (2200), respectively connected to a first micro-projector (3100) and a second micro-projector (3200), where the cell-phones are the set of processing means capable of storing, capturing, showing, communicating and/or transmitting images or video, and the micro-projectors are capable of receiving image or video data from the processing means and conform an image to be shown on the projection screen (1000). As shown in FIG. 8, a patient (6000) is laying on a surgery table (5000) where the projection screen (1000) of the invention was placed, and the micro-projectors (3100 and 3200) are hung respectively from medical device holders (4100 and 4200) which also have fixed cell-phone holders (not shown in figures) and where the cell-phones (2100 and 2200) may be respectively connected by cable to their respective micro-projector (3100 and 3200).

The projection system that includes the projection screen under the principles of the present invention reduces anxiety and the level of confinement of a person subject to surgery or medical procedures when the system is in use and allows the person to be aware of the environment surrounding such person.

The effects of such anxiety reduction will be better understood in connection with the following examples, which are intended only for enabling a full understanding of the specific embodiments herein described, but those skilled in the art shall understand that based on the detailed description and the principles of the invention as described, numerous variations are possible for other embodiments that will encompass the same principles. Therefore, the scope of the invention shall not be limited by the drawings or examples but by the appended claims.

### EXAMPLES

### Example. Cesarean Delivery

A Group A of 35 mothers giving birth by cesarean delivery, who did not use any type of virtual support therapy, were measured anxiety levels as a pain measurement in a scale of 1 to 10 using EVA Scale. In order to control anxiety, the patients were given phentanyl and midazolam and did not watch their babies. The corresponding average anxiety level is shown in Table 1.

Another Group B of 20 patients subject to cesarean delivery was treated for anxiety using the projection of virtual reality scenarios on a head mounted display, where mothers did not watch their babies. In order to control anxiety, the patients were not given phentanyl and midazolam. The average level of anxiety is also shown in Table 1.

A third Group of 34 patients 2C subject to cesarean delivery was treated for anxiety using the projection screen and system of the present invention in the embodiment shown in the figures, where they watched their relatives and the newly born in real time. In order to control anxiety, the patients were not given phentanyl and midazolam. The average level of anxiety is also shown in Table 1.

TABLE 1
Average anxiety of different of patients subject
to different surgeries or medical procedures

| | | | Average anxiety |
|---|---|---|---|
| Group | Anxiety treatment | Surgery | EVA scale |
| A | Regional anesthesia + | Cesarean | 8.5 during cesarean |
| | Phentanyl and midazolam | delivery | |
| B | Virtual Reality headset + | Cesarean | 5.3 during cesarean |
| | Regional anesthesia | delivery | |
| C | Projection screen of the | Cesarean | 2.95 After mothers |
| | invention + Regional | Delivery | watch their babies |
| | anesthesia | | |

As it can be observed, the virtual headset of the prior art did help patients to reduce anxiety by 37% in the case of cesarean delivery, whereas the projection screen of the present invention had a very large impact in reducing anxiety levels in mothers giving birth by cesarean delivery by 65% with respect to the drug methods of the prior art. There was also measured a 44% of difference between the groups with the headset and the projection screen of the invention, thereby demonstrating that there is a further anxiety reduction as compared to other image projection systems of the prior art, which would not allow to show the mothers both, their relatives and their babies in a non-confined environment.

According to the present description, it shall be observed by those skilled in the art that the projection screen and the system of the present invention have been designed to allow the surgery or medical team to perform safely and efficiently the tasks while the patient subject to a surgery or medical team has access to images that will allow to improve its confidence and psychological and physiological behavior, and it will be apparent for those skilled in the art that the embodiments illustrated have many possible variations within the scope of the principles described, and that therefore, such embodiments shall not be construed as limitative of the present invention, which should be limited only by the features of the appended claims.

1. A projection screen comprising:
a laminar body with an arched surface for projection of images that is configured to face the concave side of the arched surface towards a patient, who will see the images therein projected, wherein the arc has a length and angle enough to define a space from the nose of the patient to the center of the arc that is enough to allow a health professional to monitor the patient's signs and symptoms and to maneuver around the patient's mouth and nose in case of emergency; and
supporting means support the laminar body and place the projection screen on a surface where the patient lays; wherein the projection screen is made in one piece, without sharp edges, of a material that is sterilizable, disinfectant and/or susceptible of disinfection, and has an opacity enough to allow the patient and the members of the surgery or medical team to see through the projection screen but also to allow the projection of images on the arched surface to be seen by the patient during surgery or medical procedures.

2. The projection screen according to claim 1, wherein the laminar body is made of a composite of at least two layers, a support layer comprising a transparent polymeric material for providing mechanical support to the projection screen, and a projection layer comprising at least one film with a surface treated to provide the opacity and texture necessary for the projection of the images.

3. The projection screen according to claim 2, wherein the laminar body comprises additionally a protective layer of transparent polymeric material thereby forming a composite where the projection layer is embedded within the support and the protective layers.

4. The projection screen according to claim 3, wherein the protective layer is made of the same material as the support layer.

5. The projection screen according to claim 3, wherein the protective layer is located on the concave side of the arched surface of the projection screen.

6. The projection screen according to claim 1, wherein the arc defining the arched surface of the projection screen has an angle from 130 to 250 degrees.

7. The projection screen according to claim 6, wherein the arc defining the arched surface of the projection screen has an angle from 180 to 250 degrees.

8. The projection screen according to claim 1, wherein the arc defining the arched surface of the projection screen, has a cord equal to the standard width of a surgery table.

9. The projection screen according to claim 1, wherein the arc defining the arched surface of the projection screen has a cord as a minimum 30% larger than the maximum expected biacromial diameter of a person.

10. The projection screen according to claim 1, wherein the supporting means are located along the edge of the laminar body at the extremes of the arch defined by its arched surface and extend along the cord of the arch, thereby joining the two arched surface extremes to conform a single piece with the laminar body and to provide a supporting surface for contact with another surface intended to let a patient lay.

11. The projection screen according to claim 10, wherein the supporting means comprise a first section that extends from the end point of the laminar body towards the supporting surface and a second section consisting of the supporting surface itself.

12. The projection screen according to claim 1, wherein the supporting means define a larger length on one side of the laminar body than the length of the supporting means at the other side of the laminar body, to provide a larger space to allow a health professional to maneuver around the mouth and nose of a patient subject to surgery or medical procedures but also to provide a better angle for the patient and the health professionals to view the projected images.

13. The projection screen according to claim 11, wherein the larger length is defined by the first section.

14. The projection screen according to claim 6, wherein the arc defining the arched surface is not perpendicular to the supporting means.

15. The projection screen according to claim 14, wherein the arc defining the arched surface at the front of the projection screen has a different angle than the arc defining the arched surface at the back of the projection screen.

16. The projection screen according to claim 1, wherein the supporting means include holding means.

17. A the projection screen according to claim 16, wherein the holding means consist of at least one handle that is formed by an opening on the side of the supporting means.

18. The projection screen according to claim 16, the supporting means include shapes to fit the arms of a patient in order to increase the comfort during surgery or medical procedures.

19. A patient assistance projection system comprising
the projection screen according to claim 1;
at least one set of processing means capable of storing, capturing, showing, communicating and/or transmitting images or video; and,
at least one projector capable of receiving image or video data from the processing means and conform an image to be shown on the projection screen.

20. The patient assistance projection system according to claim 19, wherein the processing means are selected from cell phones or tablets with image and video capture and transmission capabilities, which are connected to live video contents or have memory stored image or video contents.

21. The patient assistance projection system according to claim 19, wherein the processing means are connected to the projector by cable or wireless communication systems.

22. The patient assistance projection system according to claim 19, wherein the projection system includes holders for the processing means and/or the projector, to facilitate manipulation and location of both during surgery or medical procedures.

23. The patient assistance projection system according to claim 22, wherein the holders are selected from cellphone holders, tripods, surgery room fixtures or medical device holders.

24. The patient assistance projection system according to claim 19, wherein the projector is a micro-projector capable of projecting from at least 1 meter to fit the projected content on the arched surface.

25. The patient assistance projection system according to claim 19, wherein the projected material is suitable to reduce the anxiety of the patient.

26. A method for reducing the anxiety level of a patient subject to surgery or a medical procedure comprising the steps of:
placing the projection screen according to claim 1;
projecting on the projection screen an image or video suitable to reduce the anxiety of the patient; and,
subjecting the patient to the surgery or medical procedure.
